(19) United States
(12) Patent Application Publication
(10) Pub. No.: US 2014/0322299 A1
(43) Pub. Date: Oct. 30, 2014

(54) Controlled Release of Active Using pH

(71) Applicant: Avery Dennison Corporation, Glendale, CA (US)

(72) Inventor: Anne Marie Wibaux, Cleveland Heights, OH (US)

(21) Appl. No.: 14/358,503

(22) PCT Filed: Nov. 14, 2012
(86) PCT No.: PCT/US2012/065014

§ 371 Date: May 15, 2014

Related U.S. Application Data
(60) Provisional application No. 61/560,938, filed on Nov. 17, 2011.

(51) Int. Cl. A61K 47/32; A61K 9/70; A61K 31/155

(52) U.S. Cl. CPC A61K 47/32; A61K 31/155; A61K 9/70
USPC 424/443; 514/635

(57) ABSTRACT

Dissolvable polymeric compositions are described which when exposed to certain pH conditions, controllably release one or more actives. The active agents can be nearly any active agent such as chlorhexidine gluconate (CHG). Also described are products using such compositions such as dressings. In addition, methods of controllably releasing one or more actives are described.

### CROSS REFERENCES TO RELATED APPLICATIONS

The present application claims the benefit of U.S. Provisional Patent Application No. 61/560,938 filed Nov. 17, 2011, which is incorporated herein by reference in its entirety.

### FIELD

The present subject matter relates to methods for controllably releasing one or more active agents from a polymeric composition to a wound or region of infection; and products for providing such methods.

### BACKGROUND

A wide array of medical products use adhesive for affixing the product onto a user's skin. As will be appreciated, it is desirable to prevent or at least minimize microbial growth or reproduction along the interface of adhesive and skin, as such can readily lead to infection and other undesirable conditions.

Accordingly, artisans have incorporated a wide range of antimicrobial agents into medical products or materials. Although a limited number of such agents have been incorporated into adhesives, effective release of such agents from an adhesive composition presents a formidable technical challenge. It is difficult to efficiently and controllably release such agents from the adhesive.

Chlorhexidine gluconate has a broad antimicrobial spectrum, is safe, and is well accepted in the market. However for a variety of reasons, it is difficult to controllably release this agent into a wound or region of infection.

Accordingly, it would be desirable to provide a method for releasing one or more agents such as chlorhexidine gluconate from a medical product such as a dressing such that the agent is controllably released to a region of interest such as a wound or other biological area. It would also be desirable to provide a composition containing chlorhexidine gluconate and which released that agent in a controlled and/or established manner.

### SUMMARY

The difficulties and drawbacks associated with previously known compositions, products, and practices are addressed in the present methods, adhesive compositions, products using such compositions and related methods.

In one aspect, the present subject matter provides a polymeric composition adapted to controllably release at least one active agent. The composition comprises at least one polymer forming a polymeric matrix, and at least one active agent dispersed in the polymeric matrix. Upon exposure to a pH of at least 6.5, the polymeric matrix dissolves thereby controllably releasing the at least one active agent from the composition.

In another aspect, the subject matter provides a method for controllably releasing at least one active agent from a polymeric composition. The method comprises providing a polymer which when formed in a polymeric matrix, dissolves upon exposure to a pH of at least 6.5. The method also comprises combining at least one active agent with the polymer. The method additionally comprises forming the polymeric matrix having the at least one active agent dispersed therein. And, the method comprises exposing the polymeric matrix to an environment having a pH of at least 6.5, whereby the at least one active agent is controllably released therefrom.

In another aspect, the present subject matter provides an article adapted for placement over a biological surface. The article comprises a substrate, and a layer of a polymeric composition disposed on at least a portion of the substrate. The polymeric composition includes a polymer providing a polymeric matrix and at least one antimicrobial agent dispersed in the polymeric matrix. Upon exposure to a pH greater than 6.5 the polymeric matrix dissolves thereby releasing the at least one antimicrobial agent.

As will be realized, the subject matter is capable of other and different embodiments and its several details are capable of modifications in various respects, all without departing from the subject matter. Accordingly, the description is to be regarded as illustrative and not restrictive.

### DETAILED DESCRIPTION OF THE EMBODIMENTS

In one aspect, the present subject matter provides a polymeric matrix that dissolves at a pH greater than about 6.5 and preferably greater than about 7.0 and releases one or more active agents. Preferably, the polymer matrix includes one or more antimicrobial agents or any agent that would be favorable to infection control or the wound healing process. It is known that upon infection, the pH of a wound area increases, typically to a value of greater than about 6.5 or 7. And so, upon application to a region of infection, the polymer matrix begins to dissolve thereby releasing the agent(s).

### Polymeric Composition

A wide array of polymers can be used in the preferred embodiment polymeric compositions. As previously noted, the polymer preferably dissolves at a pH at or above 6.5, and preferably at or above a pH of 7. Such polymers can be based on lactic acid. Another class of suitable polymers are pharmaceutical grade polymers typically used in time delayed delivery products to preclude release of a pharmaceutical agent in the stomach. These polymers are widely available under the designations EUDRAGIT grade polymers available from Evonik Industries. Specific examples include EUDRAGIT S100, EUDRAGIT S12.5 and EUDRAGIT FS30D. EUDRAGIT S100 and S12.5 polymers are anionic copolymers based on methacrylic acid and methyl methacrylate in which the ratio of free carboxyl groups to ester groups is about 1:2, respectively. The weight average molecular weight (Mw) of EUDRAGIT S100 and S12.5 polymers is about 125,000 g/mol. EUDRAGIT FS30D material is an aqueous dispersion of an anionic copolymer based on methyl acrylate, methyl methacrylate, and methacrylic acid in which the ratio of free carboxyl groups to ester groups is about 1:10, respectively. The weight average molecular weight (Mw) of EUDRAGIT FS30D is about 280,000 g/mol. Generally, dissolvable pharmaceutical grade polymers are based upon anionic polymers with methacrylic functional groups.

The polymeric composition preferably includes one or more active agents such as chlorhexidine gluconate (CHG). The active(s) are preferably hydrophilic. The active(s) are preferably in solid form to promote processing and incorporation in the polymeric composition. The active(s) are also preferably soluble in saline so that upon exposure to a wound or region of infection, the active can diffuse thereinto.

The active in solid form can be nearly any shape. Non-limiting examples of such shapes include flake, spherical, or a relatively thin film. When provided in a film form, the film of active is in certain embodiments, separated from the wound or region of infection by a layer or thin film of polymer.

The polymeric composition is preferably an adhesive composition. This refers to compositions that exhibit adhesive properties or characteristics. A preferred example of an adhesive composition is a pressure sensitive adhesive. A wide array of adhesive compositions are known in the art. A preferred type of adhesive is an acrylic adhesive.

### Chlorhexidine

Chlorhexidine is a chemical antiseptic and generally used as an antimicrobial agent. It is effective on both Gram-positive and Gram-negative bacteria, although it is less effective with some Gram-negative bacteria. It has both bactericidal as well as bacteriostatic mechanisms of action, the mechanism of action being membrane disruption and not ATPase inactivation as previously thought. It is also useful against fungi and enveloped viruses, though this has not been extensively investigated. Products containing chlorhexidine in high concentrations should be kept away from eyes and the ears, due to the risk of damage to those organs. However, chlorhexidine is safely used in very low concentrations, for example in some contact lens solutions.

Chlorhexidine gluconate (also known as chlorhexidine digluconate) is a salt of chlorhexidine and gluconic acid. The structural formula of chlorhexidine gluconate is:

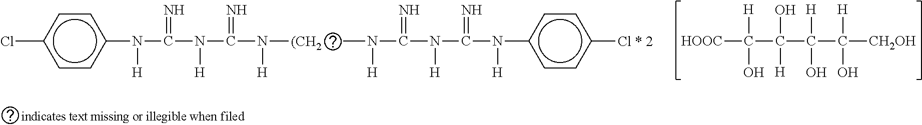

Although this compound is actually a digluconate compound, it is commonly referred to as chlorhexidine gluconate.

Thus, the term chlorhexidine gluconate as used herein encompasses the digluconate compound. Also, the terms “chlorhexidine gluconate” and “chlorhexidine digluconate” are used interchangeably herein.

Pharmaceutically acceptable chlorhexidine salts that may be used as antimicrobial agents according to the subject matter include, but are not limited to, chlorhexidine palmitate, chlorhexidine diphosphanilate, chlorhexidine dihydrochloride, chlorhexidine diacetate, and chlorhexidine digluconate. Chlorhexidine free base is a further example of an antimicrobial agent.

Thus, the present subject matter provides methods for incorporating one or more chlorhexidine salts and particularly chlorhexidine gluconate in a solvent based adhesive such as an acrylic adhesive. Although the present subject matter is particularly directed to the incorporation of chlorhexidine gluconate, the subject matter is applicable to other chlorhexidine salts and related compounds. Generally, any chlorhexidine salt that is generally provided or produced in an aqueous or liquid form is a candidate for the various preferred aspects of the subject matter as described herein.

### One or More Active(s)

As previously noted, a wide array of active agents can be incorporated into the polymeric composition. A preferred type of active for incorporation in the polymeric composition is an antimicrobial agent. A particularly preferred antimicrobial agent is one or more chlorhexidine salts and most preferably chlorhexidine gluconate (CHG). Additional examples of other antimicrobial agents include but are not limited to polyhexamethylene biguanide (PHMB), chlorhexidine salts such as chlorhexidine gluconate (CHG), silver, and combinations thereof. It will be appreciated that in addition to, or instead of an antimicrobial agent, additional additives can be incorporated in the polymeric material.

### Additional Additives

One or more additional additives can be incorporated into the polymeric composition and/or chlorhexidine formulation. Preferably the additional additives include medicinal compounds. Such medicinal compounds include, but are not limited to, antimicrobials, antibiotics, antifungal agents, antiviral agents, antithrombogenic agents, anesthetics, anti-inflammatory agents, analgesics, anticancer agents, vasodilation substances, wound healing agents, angiogenic agents, angiostatic agents, immune boosting agents, growth factors, and other biological agents. Suitable antimicrobial agents include, but are not limited to, biguanide compounds; triclosan; penicillins; tetracyclines; aminoglycosides, such as gentamicin and Tobramycin™; polymyxins; rifampicins; bacitracins; erythromycins; vancomycins; neomycins; chloramphenicols; miconazole; quinolones, such as oxolinic acid, norfloxacin, nalidixic acid, pefloxacin, enoxacin, and ciprofloxacin; sulfonamides; nonoxynol 9; fusidic acid; cephalosporins; and combinations of such compounds and similar compounds. The additional antimicrobial compounds provide for enhanced antimicrobial activity. Another preferred active is one or more MMP inhibitors.

### Methods and Incorporation of Active(s) in Polymeric Composition

The one or more active(s) are incorporated in the polymer by a wide array of known techniques such as by blending, mixing, stirring or the like.

Typically, the weight proportion of active to polymer is at least about 0.1% to about 90%, and preferably from about 0.1% to about 10%.

### Articles

The present subject matter also provides various articles that include the preferred embodiment compositions described herein. The articles can be in a variety of different forms and configurations. Preferably the articles are medical articles adapted for covering a biological surface such as for example a wound or other region undergoing healing. Non-limiting examples of such medical articles include dressings, wound coverings, and bandages. Preferably, the article comprises a substrate such as a fabric, mesh, or film. The article also comprises one or more layers or regions of a polymeric composition disposed on at least a portion of the substrate. The polymeric composition includes a polymer as described herein that provides a polymeric matrix and at least one antimicrobial agent dispersed in the polymeric matrix. Upon exposure to a pH greater than 6.5 the polymeric matrix dissolves thereby releasing the at least one antimicrobial agent.

Many other benefits will no doubt become apparent from future application and development of this technology.

All patents, published applications, and articles noted herein are hereby incorporated by reference in their entirety.

It will be understood that any one or more feature or component of one embodiment described herein can be combined with one or more other features or components of another embodiment. Thus, the present subject matter includes any and all combinations of components or features of the embodiments described herein.

As described hereinabove, the present subject matter solves many problems associated with previous type products, adhesives and practices. However, it will be appreciated that various changes in the details, materials and arrangements of components and operations, which have been herein described and illustrated in order to explain the nature of the subject matter, may be made by those skilled in the art without departing from the principle and scope of the subject matter, as expressed in the appended claims.

1. A polymeric composition adapted to controllably release at least one active agent, the composition comprising:
at least one polymer forming a polymeric matrix;
at least one active agent dispersed in the polymeric matrix;
wherein upon exposure to a pH of at least 6.5, the polymeric matrix dissolves thereby controllably releasing the at least one active agent from the composition.

2. The polymeric composition of claim 1 wherein the at least one active agent is hydrophilic.

3. The polymeric composition of claim 1 wherein upon exposure to a pH of at least 7.0, the polymeric matrix dissolves.

4. The polymeric composition of claim 1 wherein the at least one active agent is an antimicrobial agent.

5. The polymeric composition of claim 4 wherein the antimicrobial agent is selected from the group consisting of chlorhexidine salt(s), polyhexamethylene biguanide (PHMB), silver, and combinations thereof.

6. The polymeric composition of claim 5 wherein the antimicrobial agent is a chlorhexidine salt.

7. The polymeric composition of claim 6 wherein the chlorhexidine salt is chlorhexidine gluconate (CHG).

8. The polymeric composition of claim 1 wherein the weight proportion of the at least one active agent to the polymer is from about 0.1 to about 90%.

9. The polymeric composition of claim 1 wherein the weight proportion of the at least one active agent to the polymer is from about 0.1% to about 10%.

10. The composition of claim 1 wherein the composition exhibits adhesive properties.

11. A method for controllably releasing at least one active agent from a polymeric composition, the method comprising:
providing a polymer which when formed in a polymeric matrix, dissolves upon exposure to a pH of at least 6.5;
combining at least one active agent with the polymer; and
forming the polymeric matrix having the at least one active agent dispersed therein; and
exposing the polymeric matrix to an environment having a pH of at least 6.5, whereby the at least one active agent is controllably released therefrom.

12. The method of claim 11 wherein the at least one active agent is hydrophilic.

13. The method of claim 11 wherein upon exposure to a pH of at least 7.0, the polymeric matrix dissolves.

14. The method of claim 11 wherein the at least one active agent is an antimicrobial agent.

15. The method of claim 11 wherein the antimicrobial agent is selected from the group consisting of chlorhexadine salt(s), polyhexamethylene biguanide (PHMB), silver, and combinations thereof.

16. The method of claim 15 wherein the antimicrobial agent is a chlorhexidine salt.

17. The method of claim 16 wherein the chlorhexidine salt is chlorhexidine gluconate (CHG).

18. The method of claim 11 wherein the weight proportion of the at least one active agent to the polymer is from about 10% to about 90%.

19. The method of claim 11 wherein the weight proportion of the at least one active agent to the polymer is about 50%.

20. An article adapted for placement over a biological surface, the article comprising:
a substrate;
a layer of a polymeric composition disposed on at least a portion of the substrate, wherein the polymeric composition includes a polymer providing a polymeric matrix and at least one antimicrobial agent dispersed in the polymeric matrix, upon exposure to a pH greater than 6.5 the polymeric matrix dissolves thereby releasing the at least one antimicrobial agent.

21. The article of claim 20 wherein the at least one active agent is soluble in saline.

22. The article claim 20 wherein upon exposure to a pH of at least 7.0, the polymeric matrix dissolves.

23. The article of claim 20 wherein the at least one active agent is an antimicrobial agent.

24. The article of claim 23 wherein the antimicrobial agent is selected from the group consisting of chlorhexidine salt(s), polyhexamethylene biguanide (PHMB), silver, and combinations thereof.

25. The article of claim 24 wherein the antimicrobial agent is chlorhexidine salt(s).

26. The polymeric composition of claim 25 wherein the chlorhexidine salt(s) is chlorhexidine gluconate (CHG).

27. The polymeric composition of claim 20 wherein the weight proportion of the at least one active agent to the polymer is from about 10% to about 90%.

28. The polymeric composition of claim 20 wherein the weight proportion of the at least one active agent to the polymer is about 50%.

29. The composition of claim 20 wherein the composition exhibits adhesive properties.
